(19) United States
(12) Patent Application Publication
(10) Pub. No.: US 2003/0012760 A1
(43) Pub. Date: Jan. 16, 2003

(54) Novel scrub cleaning agents for the skin and novel cosmetic compositions

(76) Inventors: Fabienne Jehn-Rendu, Cedex (FR); Bruno Sabot, Cedex (FR)

Correspondence Address: ARENT FOX KINTNER PLOTKIN & KAHN, 1050 CONNECTICUT AVENUE, N.W., SUITE 400, WASHINGTON, DC, 20036, US

(21) Appl. No.: 10/204,415

(22) PCT Filed: Feb. 28, 2001
(86) PCT No.: PCT/FR01/00616

(30) Foreign Application Priority Data
Feb. 28, 2000 (FR) 00/02664

(51) Int. Cl. A61K 7/06; A61K 7/11

(52) U.S. Cl. 424/70.15; 424/70.16

(57) ABSTRACT

The invention concerns a cosmetic or dermatological composition comprising at least a superabsorbent (co)polymer. The resulting composition exhibits remarkable skin cleansing scrub activity and has no abrasive effect.

This invention relates to the technical area of cosmetic compositions and more specifically “cleansing agents” or cosmetic products permitting elimination of elements of dead body or facial skin which accumulate on the surface of the epidermis.

The word “superabsorbent” appearing throughout this application is to be understood to mean a reticulated polymer or copolymer capable of absorbing moisture or an aqueous solution or a water-alcohol solution. The amount of moisture or solution absorbed may represent up to 1000 times the initial volume of the superabsorbent.

Superabsorbents or “SAP” and their absorptive properties are very well known to the expert; hence it is pointless to specify them in detail here.

If he so desires, the expert may refer to the following patents selected from among a large number of known patents and documents.

U.S. Pat. No. 3,669,103 A slightly reticulated polymer such as poly-N-vinylpyrrolidone, polyvinyltoluenesulfonate, poly-2-hydroxyethyl acrylate, polyvinylmethyloxazolidone, hydrolyzed polyacrylamide, polyacrylic acid, copolymers of acrylamide and acrylic acid, and alkaline metal salts of polymers such as contain sulfonate or carboxy groups, in particular partially hydrolyzed, slightly reticulated polyacrylamide.

FR 2 559 158 reticulated polymers of acrylic or methacrylic acid, grafted reticulated copolymers such as polysaccharide/acrylic or methacrylic acid, reticulated terpolymers such as acrylic or methcrylic acid/acrylamide/sulfonated acrylamide, and their alkaline-earth metal or alkaline salts.

The chief characteristic of these polymers is a high capacity for swelling in an aqueous medium. They are used especially in agriculture to retain moisture in soils, in chemistry, and in industrial processes, in absorbent sanitary products, in hygienic products for infants designed to contain urine, and in similar applications.

Their known applications are associated with their absorbent properties

An essential problem in connection with use of skin cleansing agents relates to the phenomenon of abrasion which occurs during the use of such agents and generally is manifested in friction and blotches on the surface of the skin.

This effect is of course extremely annoying, above all in the area of cosmetic products considered, since it results in replacement of one skin imperfection by another and more punishing one.

Consequently, there is a very great need in industry for skin cleansing agents which might perform the function assigned to them but without the serious disadvantages such as those referred to above.

It is claimed for this invention that it has surprisingly been discovered that reticulated polymers of the superabsorbent family may be used in cosmetic or dermatological compositions as efficient cleansing agents which do not exhibit such disadvantages.

This invention relates to cosmetic or dermatological compositions effecting cleansing action characterized in that the compositions comprise at least one superabsorbent (co)polymer.

It is claimed for the invention that superabsorbent polymers may be incorportated into formulations of various kinds such as aqueous solutions, gel-cremes, emulsions, and similar formulations known to the expert.

In one preferred embodiment the compositions are characterized in that they represent at least 0.01 to 5 percent by weight of the total composition and comprise at least one superabsorbent polymer as skin cleansing agent.

The superabsorbents which may be used are reticulated homopolymers or copolymers of the moisture-retaining type whose structure is well known to the expert and which are based in particular on:

one or more of the monomers described below:

Non-ionic monomers: acrylamide, methacrylamide, N-vinyl pyrrolidone, vinyl acetate, vinyl alcohol, acrylate esters, allyl alcohol

Monomers performing a carboxylic function: acrylic acid, methacrylic acid, and their salts

Monomers performing a sulfonic acid function: 2-acrylamido-2-methyl propane sulfonic acid (AMPS) and their salts

Cationic monomers such as dialkylaminoalkyl (meth)acrylate, dialkylaminoalkyl (meth)acrylamide, diallylamine, methyldiallylamine, and their quaternary or acid salts

and one or more of the reticulates described below, such as methylene bisacrylamide (MBA), ethylene glycol diacrylate, polythylene glycol dimethacrylate, diacrylamide, cyanomethylacrylate, vinyloxyethylacrylate or mthacrylate and formaldehyde, glyoxal, compounds of the diglycidylether family such as ethyleneglycoldiglycidylether, or epoxys, or metal salts, or any other means well known to the expert permitting reticulation.

In one preferred embodiment the superabsorbents are to be selected from among reticulated polyacrylate or polyacrylamides.

In one preferred application the compounds claimed for the invention are characterized in that the amount of the reticulation agent is greater than 50 or 100 ppm, preferably greater than 300 ppm, relative to the polymer and preferably ranges from 50 to 1000 or 10,000 ppm, relative to the polymer or more for methylenebisacrylamide (MBA) for equivalent reticulation effected with a reticulation agent of identical or different efficiency.

Very large reticulation amounts may be used since, while not wishing to be bound to any theory, the Applicant does believe that the function of the SAP used as claimed for this invention is in effect not the same as the traditional function of SAPs or has been modified in a way which has not been clarified.

In one particular embodiment the superabsorbents tested for the tests are ones marketed under the FLOCARE™ brand name by the SNF SA company.

In one other particular embodiment, the cleansing agent claimed for the invention consists of a mixture of a plurality of the superabsorbent polymers described in the foregoing.

The amount and nature of the superabsorbent polymers selected vary with:

the intended cosmetic effect: very regular or punctual use of the composition

the area of application: face, bust, body, feet, . . .

the condition of the skin: dry, oily, mature, sensitive, . . .

the cosmetic formulation as a whole: the superabsorbent may be used in particular with other ingredients exerting similar and/or complementary effects.

The invention also relates to cleansing cosmetic compositions comprising, in addition to the one or more superabsorbent polymer(s) such as those described above, other ingredients exhibiting properties of cleansing by mechanical action and/or by chemical action, thereby making it possible to achieve synergy of effects.

The invention also relates to cosmetic cleansing compositions such as those described in the foregoing and also comprising other known ingredients used for cosmetic purposes and of cosmetic quality.

The expert will be able to prepare the appropriate compositions as a function of the parameters indicated above and the examples provided in this application. The cleansing agents claimed for the invention may be used in particular in various types of cosmetic compositions, such as aqueous solutions (liquid soap, gel creme, . . . ), emulsions, etc.

In addition, the pH value of the final formulations may range from 1 to 13, preferably from 3 to 8, without affecting the stability of the superabsorbent polymer.

The invention also relates to the various processes for preparation of the compositions claimed for the invention, processes in which the superabsorbent polymer(s) used as cleansing agent are mixed with a suitable base in keeping with the nature of the final composition, by a method known to the expert.

The invention relates especially to a non-restrictive process for preparation of a cleansing gel/cream, one by means of which the aqueous phase is prepared with water and preservatives; the superabsorbent polymer (in particular the polymer FLOCARE™ DP/JB 2024) is added to the aqueous phase, and, after homogenizing and complete inflation of the superabsorbent polymer, FLOCARE™ ET 100 is added.

The invention also relates to a non-restrictive process for preparation of an oil/water plus cleansing cream base for the face, a process in which the aqueous phase comprising water and preservatives is prepared; the superabsorbent polymer (in particular the polymer FLOCARE™ DP/JB 2024) is added during agitation to the aqueous phase, and, after homogenizing, the oily phase is incorporated and FLOCARE™ ET100 is added.

The invention also relates to a non-restrictive process for preparation of a cleansing cream-based oil/water emulsion for the body, a process whereby the aqueous phase with water and preservatives is prepared; the superabsorbent polymer (the polymer FLOCARE™ DP/SB 2061 in particular) is added to the aqueous phase during agitation and, after homogenizing, the oily phase is incorporated and then FLOCARE™ ET100 is added.

The results of various tests conducted are recapitulated in the following examples, along with the properties of the compositions obtained.

NOTES

The superabsorbent polymer designated as FLOCARE™ DP/JB 2024 is a reticulated acrylic acid/sodium acrylate copolymer of a grain size below 300 microns.

The superabsorbent polymer designated as FLOCARE™ DP/SB 2061 is a reticulated acrylic acid/sodium acrylate copolymer of a grain size of 100 to 800 microns.

The polymer FLOCARE™ ET 100 is a sodium polyacrylate used as a thickening agent and/or emulsifier.

The names of the ingredients used in the compositions are those referenced in the INCL (International Nomenclature of Cosmetic Ingredients), after translation if applicable.

### EXAMPLES

EXAMPLE 1
base for gel/cleansing cream

| | percent by weight |
|---|---|
| FLOCARE ™ DP/JB 2024 | 0.2% |
| Water (aqua) | QSP [sufficient amount to make up] 100 [%] |
| FLOCARE ™ ET100 | 1% |
| preservatives | QS [sufficient amount] |
| perfume | QS |

Procedure:

The aqueous phase comprising water and preservatives is prepared. The superabsorbent polymer (FLOCARE™ DP/JB 2024) is added to the aqueous phase during agitation. FLOCARE™ ET100 is added after homogenizing and full expansion of the superabsorbent polymer.

Final Characteristics of the Emulsion:

translucid appearance of composition

grains of superabsorbent polymers visible; they are very readily felt when applied

composition easy to rinse off

EXAMPLE 2
oil/water cleansing cream based emulsion for the face:

| | percent by weight |
|---|---|
| FLOCARE ™ DP/JB 2024 | 0.2% |
| Water (aqua) | QSP 100 |
| Octyl stearate (ethyl-hexyl stearate) | 10% |
| FLOCARE ™ ET100 | 0.7% |
| Preservatives | QS |
| Perfume | QS |

Procedure:

The aqueous phase with water and preservatives is prepared. The superabsorbent polymer (FLOCARE™ DP/JB 2024) is added to the aqueous phase during agitation. After homogenizing, the oil phase is incorporated and FLOCARE™ ET100 is then added.

Final Characteristics of Composition:

opaque, creamy appearance

grains of superabsorbent polymers visible on application

composition easy to rinse off

EXAMPLE 3
Oil/water emulsion based on cleansing cream for the body

| | percent by weight |
|---|---|
| FLOCARE ™ DP/SB 2061 | 0.4% |
| Water (aqua) | QSP 100 |
| Octyl stearate (ethyl-hexyl stearate) | 10% |
| FLOCARE ™ | 0.7% |
| Preservatives | QS |
| Perfume | QS |

Procedure:

The aqueous phase with water and preservatives is prepared. The superabsorbent polymer (FLOCARE™ DP/JB 2061) is added to the aqueous phase during agitation. After homogenizing, the oil phase is incorporated and FLOCARE™ ET100 is then added.

Final Characteristics of Composition:

opaque, creamy appearance

grains of superabsorbent polymers larger and accordingly more perceptible during application

composition easy to rinse off

### Results

Tests were conducted with volunteers for each of the cleansing bases obtained as indicated in the foregoing (examples 1, 2, and 3). The following results were obtained:

the skin is softer after application of the composition

no redness or roughness of the skin observed after application

each of the compositions is very easy to rinse off.

The expert will know how to prepare other cleansing-effect cosmetic or dermatological compositions using at least one SAP as described in the foregoing, on the basis of other SAPs and other cosmetic or dermatological ingredients.

1. Cleansing-effect cosmetic or dermatological compositions, characterized in that such compositions comprise at least one superabsorbent (co)polymer as skin cleansing agent.

2. Compositions as specified in claim 1, wherein such compositions are present in the form of various types of formulations such as aqueous solutions, gel-creams, emulsions, and the like.

3. Compositions as specified in claim 1 or 2, wherein such compositions comprise at least 0.01 to 5 percent by weight of the total composition of at least one superabsorbent polymer as skin cleansing agent.

4. Compositions as specified in any of claims 1 to 3, wherein the usable superabsorbents are reticulated homopolymers or copolymers of the type based on water-retention substances, in particular
one or more of the monomers described in the following:
non-ionic monomers: acrylamide, methacrylamide, N-vinyl pyrrolidone, vinyl acetate, vinyl alcohol, acrylate esters, allyl alcohol
monomers performing a carboxyl function: acrylic acid, methacrylic acid, and their salts.
monomers performing a sulfonic acid function: sulfonic 2-acrylamido-2-methylpropane (AMPS) and their salts.
cationic monomers such as dialkylaminoalkyl (meth)acrylate, dialkylaminoalkyl (meth)acrylamide, diallylamine, methyldiallylamine, and their quaternary ammonium or acid salts.
and one or more of the reticulants described in the following, such as methylene bisacrylamine (MBA), ethylene glycol diacrylate, polyethylene glycol dimethacrylate, diacrylamide, cyanomethylacrylate, vinyloxyethylacrylate or methacrylate and formaldehyde, glyoxal, compounds of the diglycidyl ether family such as ethyleneglycoldiglycidether, or epoxys, or metal salts, or any other means well known to the expert which permit reticulation.

5. Compositions as specified in any of claims 1 to 4, wherein the superabsorbents are selected from among reticulated polyacrylates or polyacrylamides.

6. Compositions as specified in any of claims 1 to 5, wherein the superabsorbents are ones marketed under the FLOCARE™ brand by the SNF SA company.

7. Compositions as specified in any of claims 1 to 6, wherein the cleansing agent claimed for the invention consists of a mixture of a plurality of the superabsorbent polymers described in any of claims 1 to 6.

8. Compositions as specified in any of claims 1 to 7, wherein the amount of reticulation agent is greater than 50 to 100 ppm, preferably greater than 300 ppm, relative to the polymer, and preferably ranges from 50 to 1000 or 10,000 ppm or more, relative to the polymer, especially from 100 to 800 ppm, relative to the polymer, or more, for methylenebisacrylamide (MBA), for equivalent reticulation effected with a reticulation agent of identical or different efficiency.

9. Compositions as specified in any of claims 1 to 8, wherein such compositions comprise, in one or more superabsorbent polymer(s) such as those described in any of claims 1 to 8, other ingredients exhibiting properties of cleansing by mechanical action and/or by chemical action.

10. Compositions as specified in any of claims 1 to 9, wherein such compositions comprise other ingredients which are known to be employed for cosmetic purposes and are of cosmetic quality.

11. Compositions as specified in any of claims 1 to 10, wherein the pH value of final formulations produced on the basis of such compositions may range from 1 to 13, preferably from 3 to 8.

12. Compositions as specified in any of claims 1 to 11, wherein the superabsorbent polymer is selected from among the following:
FLOCARE™ DP/JB 2024, which is a reticulated acrylic acid/sodium acrylate of a grain size below 300 microns.
FLOCARE™ DP/SB 2061, which is a reticulated acrylic acid/sodium acrylate of a grain size of 100 to 800 microns.
